(19) United States
(12) Patent Application Publication
(10) Pub. No.: US 2004/0097937 A1
(43) Pub. Date: May 20, 2004

(54) Orthopedic bone plate

(76) Inventors: Sandi Pike, Warsaw, IN (US); Jeffery A. VanDiepenbos, Syracuse, IN (US)

Correspondence Address: Jacque R. Wilson, Esq., Zimmer, Inc., P.O. Box 708, Warsaw, IN, 46580, US

(21) Appl. No.: 10/299,437

(22) Filed: Nov. 19, 2002

(51) Int. Cl. A61B 17/56

(52) U.S. Cl. 606/69

(57) ABSTRACT

An orthopedic bone plate for use in treating fractured bones. The bone plate includes at least one tapered end, a plurality of evenly spaced bores disposed along the longitudinal axis of the plate, and a plurality of recesses disposed on one side of the plate.

### BACKGROUND OF THE INVENTION

1. Field of the Invention

The present invention relates generally to orthopedic prosthetic devices. More specifically, the invention relates to orthopedic bone plates attachable to bone for the treatment of bone fractures.

2. Description of the Related Art A bone plate is typically used to maintain different parts of a fractured or otherwise severed bone substantially stationary relative to each other during and/or after the healing process in which the bone mends together. Bones of the limbs include a shaft with a head at either end thereof. The shaft of the bone is generally elongated and of relatively cylindrical shape.

It is known to provide a bone plate which attaches to the shaft or head and shaft of a fractured bone to maintain two or more pieces of the bone in a substantially stationary position relative to the one another. Such a bone plate generally comprises a shape having opposing substantially parallel sides and a plurality of bores extending between the opposing sides, wherein the bores are suitable for the receipt of pins or screws to attach the plate to the bone fragments.

Conventional bone plates, as described above, have a shape corresponding to a shape of an average bone based upon statistical data. In essence, such plates provide a roadmap for the surgeon to reconstruct the bone or place fragments of the bone against the bone plate during the reconstruction. It is quite common for the curvature of the plate to not exactly correspond to the curvature of the bone.

It is common practice with a bone plate as described above for an orthopedic surgeon to place such a bone plate against the bone, observe the differences in curvature between the bone plate and bone, remove the bone plate and bend the bone plate using various fitting methods commonly known in the art to better fit the bone, and again place the bone plate against the bone. This process is repeated until a satisfactory fit is achieved between the bone plate and the bone.

When known plates are bent to conform to a bone, such bending generally occurs “sharply” over the bores provided in the plate for receiving screws. The sharp bending occurs over the bores because they represent the weakest part of the plate. Sharp sudden bends, however, rather than gradual curves, may result in unwanted high stress areas in the bone plate.

Therefore, a need exists for a bone plate that is conformable to the shape of bone without sharply bending the plate.

In addition, the bores known in the prior art generally allow a limited degree of screw angulation, which, in turn, limits the ability of the bone screws used with such plates to capture significantly displaced bone fragments. Thus, a need exists for plates that accommodate a greater degree of bone screw angulation relative to the plate.

It is further known, that it is desirable in any surgical procedure to cause as little trauma to the patient as possible. Accordingly, surgeons have been attempting to insert implants, such as bone plates, through smaller, less invasive incisions. However, the bone plates known to those of skill in the art were not designed to accommodate such a goal. Consequently, it is difficult for surgeons to insert bone plates into the very small incisions that are employed during less invasive or minimally invasive surgeries.

Thus, a further need exists for a bone plate that can be more easily inserted through a less invasive or minimally invasive incision and attached to a fractured bone.

### SUMMARY OF THE INVENTION

The present invention provides a malleable orthopedic bone plate having a plurality of thinned portions along the plate. These thinned portions result in a plate having more uniform strength along its length. Thus, the plate can accommodate more subtle curves and an improved anatomical approximation for the bone fragments to which the plate attaches. In addition, these more subtle curves result in a plate having lower residual stress.

The present invention also provides a means for increased longitudinal and transverse screw articulation. The present invention further provides an orthopedic bone plate insertable into a relatively small incision, such as those used in less or minimally invasive surgical procedures.

The invention comprises, in one form thereof, an orthopedic bone plate for attachment to two or more pieces of a fractured bone. The bone plate includes an elongated shape having an upper side, a lower side, and a longitudinal axis. A series of bores that extend between the upper and lower surface are disposed along the longitudinal axis. In addition, a series of recesses is disposed on the upper surface of the bone plate, thereby providing several thinned portions of the bone plate across which portions the plate is more malleable than prior art plates of thickness equal to the unthinned portion of the plate. The invention further comprises at least one tapered end that enables a person to insert the bone plate into an incision suitable for a less invasive or minimally invasive surgical procedure.

An advantage of the present invention is that an improved anatomical approximation of the bone plate is achieved through deflection of the bone plate, thereby resulting in superior bone reduction at the fracture.

Another advantage of the present invention is that the plate can be smoothly bent in order to conform to bone.

A further advantage is that the improved anatomical approximation results in an increased contact interface between the bone plate and bone, resulting in more loading on the bone and less loading on the bone plate with a reduced possibility of fatigue failure of the bone plate.

A further advantage is that the superior reduction of the bone results in improved loading between the bone pieces at the fracture site, resulting in improved healing.

An additional advantage is that the plate generally experiences lower residual stresses, thereby inhibiting plate fracture.

Another advantage of the present invention is that it accommodates greater screw angulation relative to the plate.

Another advantage of the present invention is that it can be used with less invasive or minimally invasive surgical procedures.

Other advantages and features of the present invention will be apparent to those skilled in the art upon a review of the appended claims and drawings.

### BRIEF DESCRIPTION OF THE DRAWINGS

The above-mentioned and other features and objects of this invention, and the manner of obtaining them, will become more apparent and the invention itself will be better understood by reference to the following description of an embodiment of the invention taken in conjunction with the accompanying drawings, wherein:

FIG. 1 is a top perspective view of an orthopedic bone plate according to the present design.

FIG. 2 is a bottom perspective view of the embodiment shown in FIG. 1.

FIG. 3 is a front elevational view of the embodiment shown in FIG. 1.

FIG. 4 is a side cross sectional view of the embodiment shown in FIG. 1.

FIG. 5 is a side view of the embodiment shown in FIG. 1.

FIG. 6 is a top perspective view of an orthopedic bone plate according to another embodiment of the present design.

FIG. 7 is a bottom perspective view of the embodiment shown in FIG. 6.

FIG. 8 is a front elevational view of the embodiment shown in FIG. 6.

FIG. 9 is a side cross sectional view of the embodiment shown in FIG. 6.

FIG. 10 is a side view of the embodiment shown in FIG. 6.

Corresponding reference characters indicate corresponding parts throughout the several views. Although the drawings represent an exemplary embodiment of the present invention, the drawings are not necessarily to scale and certain features may be exaggerated to better illustrate and explain the invention. The exemplification set out herein illustrates an exemplary embodiment of the invention only and such exemplification.

### DETAILED DESCRIPTION OF THE INVENTION

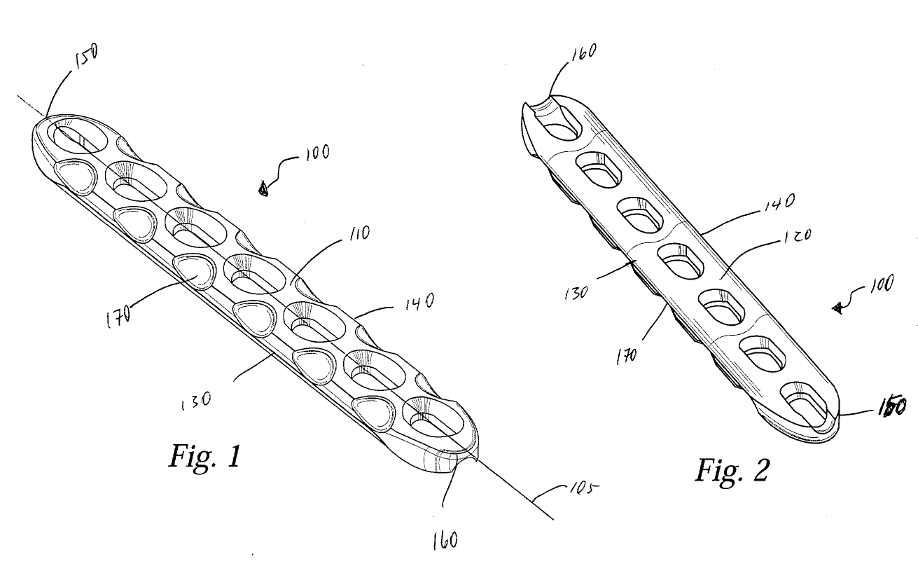

Referring initially to FIGS. 1 and 2, there is shown an orthopedic bone plate 100 according to the present invention. Bone plate 100 comprises a biocompatible polymer or biocompatible metal, such as stainless steel. Plate 100 also comprises a generally rectangular shape when viewed from the side, having longitudinal axis 105. Plate 100 further comprises an upper surface 110 and a lower surface 120 (the bone contacting surface). In the preferred embodiment, surfaces 110 and 120 are substantially parallel to one another.

Referring now to FIG. 1, exclusively, there is shown a top perspective view of plate 100. Plate 100 comprises a pair of opposing right and left sides, 130 and 140, respectively. Right and left sides 130 and 140 are preferably straight and substantially parallel to one another. Plate 100 also comprises opposing front and rear edges, 150 and 160, respectively. Edges 150 and 160 may be of any desired shape, flat, pointed, etc. In the embodiment shown, edges 150 and 160 are curved across longitudinal axis 105.

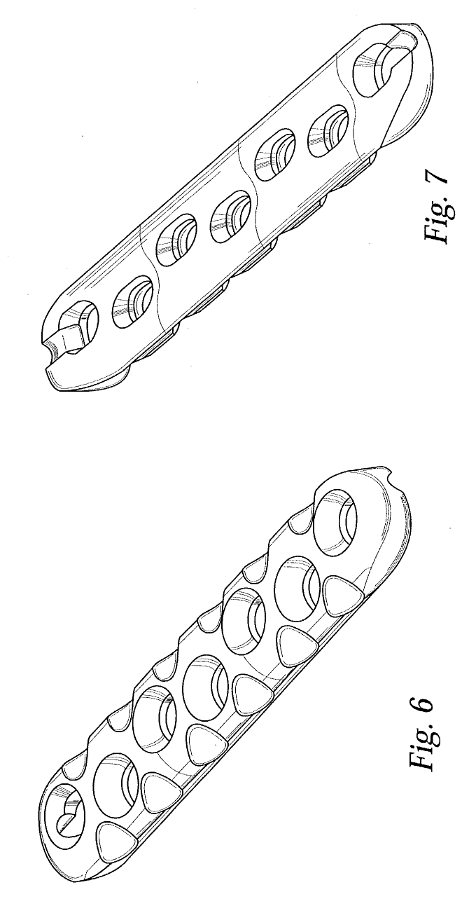

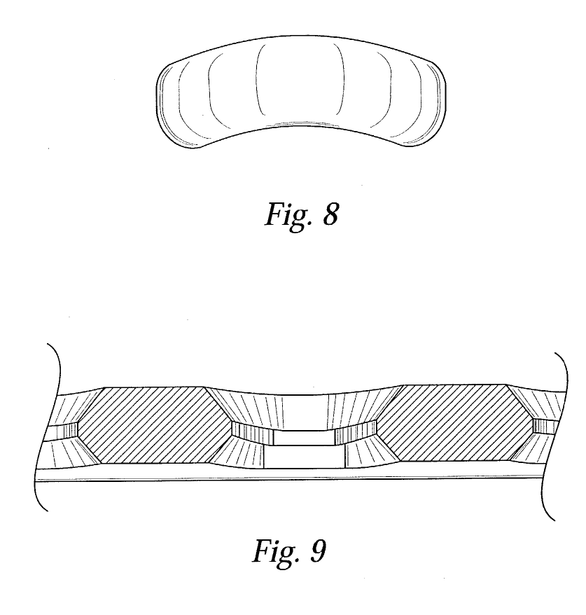

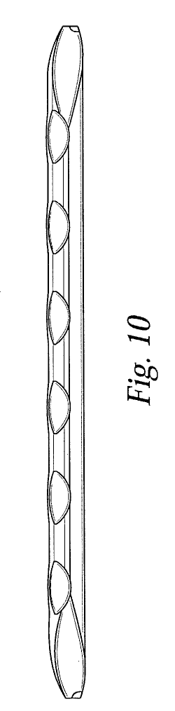

Referring still to FIG. 6, there is shown a plurality of recesses 170 disposed on upper surface 110 of plate 100. Each recess comprises a concave dish having generally curved edges disposed on surface 110. In the embodiment shown, a first column of recesses 170 is disposed on surface 110 such that each recess is in contact with left side 130. A second column of recesses 170 is disposed on surface 110 such that each recess of the second column is in contact with right side 140. The first and second columns are generally parallel to longitudinal axis 105, and recesses 170 are disposed on surface 110 such that each recess 170 of the first column directly opposes a corresponding recess 170 on the second column along a line 106 that is perpendicular to longitudinal axis 105. These multiple pairs of opposing recesses 170 are evenly distributed along the length of plate 100.

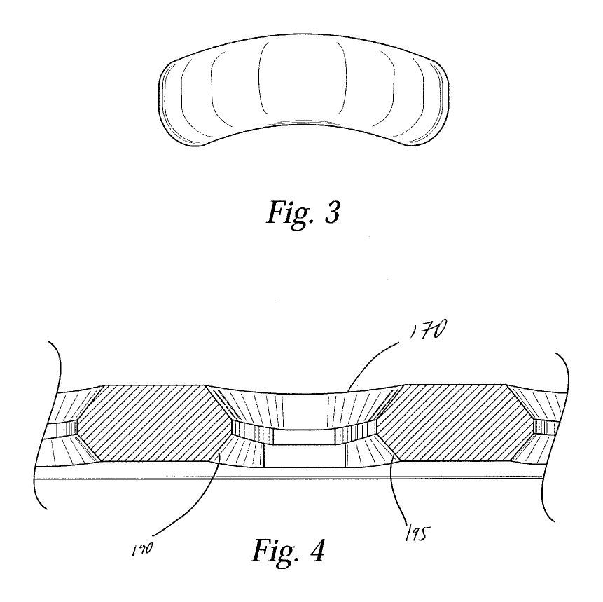

Referring now to FIG. 3, there is shown front view of an orthopedic bone plate 100 according to the present invention, wherein upper surface 110 of plate 100 is curved such that it forms a convex surface about longitudinal axis 105, and lower surface 120 is curved about longitudinal axis 105 such that lower surface 120 forms a concave surface relative to the same. Recesses 170 disposed on upper surface 110 of plate 100 create a plurality of “thinned portions” of plate 100 that transverses plate 100 perpendicularly to longitudinal axis 105. In these thinned portions, plate 100 is more malleable than in unthinned portions of plate 100. Plate 100, in the thinned portions, is bendable within a plane that is perpendicular to upper surface 110 along axis 105. The thinned portions of plate 100 are weaker than unthinned portions thereof such that a bending torque applied to plate 100 will cause a curved bend about such thinned portions and bores rather than an angled or sharp bend about specific bores.

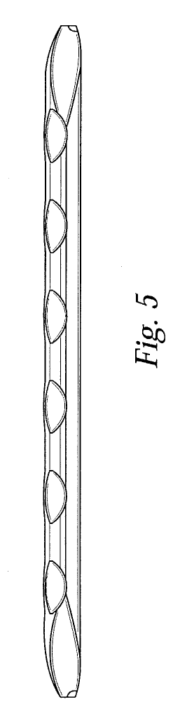

Referring now to FIG. 5, there is shown a side view of an orthopedic bone plate according to the present invention. Front edge 150 and rear edge 160 comprise a tapered shape in the plane perpendicular to upper surface 110 and lower surface 120, such that edges 150 and 160 have a depth that is less than the depth of the unthinned portions of the plate 100.

Referring now to FIG. 4, there is shown a side cross-sectional view of a bone plate according to the present invention, wherein plate 100 further comprises a plurality of bores 180 disposed through plate 100 such that each bore 180 is in communication with both upper surface 110 and lower surface 120. Each bore 180 is preferably generally perpendicular to upper and lower surfaces 150 and 160, and, as shown in FIG. 1 and FIG. 2, it is preferred that bores 180 are disposed evenly along longitudinal axis 150 such that they are disposed directly on longitudinal axis 105 or in a line adjacent to and parallel with longitudinal axis 105. In the embodiment shown, bores 180 comprise “dual compression” screw bores that are commonly known in the art such that plate 100 is attachable to bone via a plurality of bone screws. Such dual compression bores accommodate bone screws in a first and second direction such that bone fragments lying generally diagonally from one another can be properly secured. In the preferred embodiment the dual compression bores of the present invention comprise undercuts 190 and 195. These undercuts allow bores 180 to accommodate screws at disposed at angles of between about 90° and 155° relative to the bone plate. Those of skill in the art will appreciate that undercuts 190 and 195 may comprise a single undercut for each bore 180, wherein the undercut completely encircles the same.

It will be appreciated by those skilled in the art that the foregoing is a description of a preferred embodiment of the present invention and that variations in design and construction may be made to the preferred embodiment without departing from the scope of the invention as defined by the appended claims.

We claim:

1. An orthopedic bone plate, the plate comprising: a shape, the shape having two opposing sides, an upper surface, a lower surface, and a longitudinal axis; and at least one pair of transversely opposing recesses disposed on the upper surface of the plate such that each recess of the at least one pair of recesses is in communication with one of the two opposing sides, thereby providing at least one thinned portions in the plate, the thinned portions having a decreased relative thickness to unthinned portions of the plate such that the plate is more malleable along the longitudinal axis across the thinned portions relative to the unthinned portions.

2. The apparatus of claim 1, further comprising a plurality of bores disposed along the longitudinal axis of the plate.

3. The apparatus of claim 2, wherein the bores are substantially perpendicular to the longitudinal axis.

4. The apparatus of clam 2, wherein the bores are equally spaced from one another.

5. The apparatus of claim 2, wherein the bores are dual compression slots.

6. The apparatus of claim 5, wherein the bores comprise at least one undercut.

7. The apparatus of claim 1, wherein the plate is attachable to the bone using a plurality of bone screws.

8. The apparatus of claim 1, wherein the plate further comprises a front edge, and a rear edge, said front edge comprising a taper.

9. The apparatus of claim 1, wherein the plate further comprises a front edge, and a rear edge, said rear edge comprising a taper.
